(19) United States
(12) Patent Application Publication
(10) Pub. No.: US 2007/0243255 A1
(43) Pub. Date: Oct. 18, 2007

(54) Multifunctional supramolecular hydrogels as biomaterials

(76) Inventors: Bing Xu, Kowloon (HK); Zhimou Yang, Kowloon (HK); Keming Xu, Kowloon (HK)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/237,498

(22) Filed: Sep. 27, 2005

Related U.S. Application Data
(60) Provisional application No. 60/613,413, filed on Sep. 28, 2004.

(51) Int. Cl. A61K 38/46; A61K 38/14; A61K 38/05; A61K 38/04; A61K 31/7048; A61K 31/7034; A61K 31/545; A61K 31/43; A61K 9/14

(52) U.S. Cl. 424/484; 514/28; 514/29; 514/8; 514/19; 514/35; 514/192; 514/200; 514/18; 424/94.6; 977/906

(57) ABSTRACT

The present invention pertains to the design and application of a supramolecular hydrogel having a three-dimensional, self-assembling, elastic, network structure comprising non-polymeric, functional molecules and a liquid medium, whereby the functional molecules are noncovalently crosslinked. The functional molecules may be, for instance, anti-inflammatory molecules, antibiotics, metal chelators, anticancer agents, small peptides, surface-modified nanoparticles, or a combination thereof. The design of the hydrogel includes: 1) modifying functional molecules to convert them into hydrogelators while enhancing or maintaining their therapeutic properties and 2) triggering the hydrogelation process by physical, chemical, or enzymatic processes, thereby resulting in the creation of a supramolecular hydrogel via formation of non-covalent crosslinks by the functional molecules. Applications of the present invention include use of the supramolecular hydrogel, for instance, as a biomaterial for wound healing, tissue engineering, drug delivery, and drug/inhibitor screening.

This application claims the benefit of U.S. Ser. No. 60/613,413, filed Sep. 28, 2004, the contents of which are incorporated herein in its entirety by reference.

Throughout this application, various references are cited and disclosures of these publications in their entireties are hereby incorporated by reference into this application in order to more fully describe the state of the art to which this invention pertains.

### BACKGROUND OF THE INVENTION

Hydrogels, formed by three-dimensional, elastic networks whose interstitial spaces are filled with a liquid, possess many useful properties (e.g., response to external stimuli, flow in response to shear force, etc.). Because of their useful properties, hydrogels have applications in many areas, such as bioanalysis, chemical sensing, food processing, cosmetics, drug delivery, and tissue engineering.

Following the successful applications of polymer-based hydrogels in biomedical engineering and the successful studies on low molecular weight organogels, supramolecular hydrogels, formed by the self-assembly of small molecules, have recently emerged as a new type of biomaterial that promises important biomedical applications (e.g., hydrogels based on the self-assembly of oligopeptides have been used as scaffolds to grow neurons). These oligopeptide-based hydrogels, however, are only mono-functional, and their cost remains high.

In contrast, the present invention pertains to a new type of supramolecular hydrogel, wherein the self-assembled nanofibers or nano-networks of functional small molecules (or entities) serve as the matrix to encapsulate water and to form the hydrogel. Additionally, these small molecules maintain their therapeutic effects even though they serve as the structural components of the supramolecular hydrogels. Because of their resemblance to the extracellular matrix, their biocompatibility, and their biodegradability, this type of hydrogel may serve as a new and general platform for diverse applications in biomedical areas, such as removal of toxics, wound healing, tissue engineering, and drug delivery.

### SUMMARY OF THE INVENTION

The present invention pertains to the general design and application of a new supramolecular hydrogel, whose self-assembled networks comprise one or more types of functional molecules (e.g., anti-inflammatory molecules, antibiotics, metal chelators, anticancer agents, small peptides, and/or surface-modified nanoparticles), as biomaterials for a range of applications, such as wound healing, tissue engineering, drug delivery, anticancer therapy, treatment of infectious diseases, drug/inhibitor screening, and removal of toxins.

The design of the supramolecular hydrogel includes: 1) modifying functional molecules to convert them into hydrogelators while enhancing or maintaining their therapeutic activities and 2) triggering the hydrogelation process by physical, chemical, or enzymatic processes, thereby resulting in the creation of a supramolecular hydrogel via formation of non-covalent crosslinks by the functional molecules. Notably, the functional molecules maintain their therapeutic effects even though they serve as the structural components of the supramolecular hydrogels.

### DESCRIPTION OF THE FIGURES

FIG. 1. An illustration of the structures of three small molecules: N-(Fluorenyl-9-methoxycarbonyl)-L-Leucine, N-(Fluorenyl-9-methoxycarbonyl)-L-Lysine, and pamidronate. N-(Fluorenyl-9-methoxycarbonyl) -L-Leucine 1 and N-(Fluorenyl-9-methoxycarbonyl)-L-Lysine 2 belong to a novel class of anti-inflammatory agents reported by Burch, et al.,¹ and 1 displays effective anti-inflammatory activity in animal models. Neither 1 nor 2 acts as a hydrogelator in a neutral aqueous solution. The addition of pamidronate (3) to the suspension of 1 and 2 leads to the formation of a hydrogel at pH=9, in which 3 acts as both a donor and an acceptor of hydrogen bonds to promote hydrogelation. In addition, 3 is a clinically-used drug and forms a stable complex with UO₂²⁺ and reduces the poison caused by the uranyl ions.

FIG. 2. (2A) The change in the weights of the mice (initial weights are normalized as 1; 0 represents deceased mice. Data are mean±SD obtained in N mice in the group, in which N=7, 7, 5 concerning the (−), (+), and healing groups, respectively. (2B) An illustration of the plausible interaction between the hydrogel and the simulated uranium wound.

FIG. 3. (3A) The molecular structures of the ligand, vancomycin 4, and the derivatives of the receptors 5, 6, and 7. (3B) The linear viscoelastic frequency sweep responses of the hydrogels of 5 and 5+4 at strain of 1%. and 0.1%, respectively. (3C) The linear viscoelastic frequency sweep responses of the hydrogels of 6, 7, 6+4, and 7+4 at 1% strain. The concentrations of 4, 5, 6, and 7 are all 30 mM.

FIG. 4. The structure of 8 and the optical image of the hydrogel of 8 (0.36 wt %) (taken by a flatbed scanner when the vial was laid horizontally).

FIG. 5. The molecular structures of the two compounds used for the formation of hydrogels and the schematic gelation process. Conditions of gelation: (i) Na₂CO₃, buffer; (ii) enzyme, 37° C.; (iii) Na₂CO₃, buffer; and (iv) enzyme, 60° C. (buffer: pH˜9.6, 50 mM of Tris-HCl plus 1 mM of MgCl₂).

FIG. 6. An illustration of the design for identifying inhibitors of an enzyme by hydrogelation.

FIG. 7. Results of activities of three inhibitors: row 1) Left to right: sol. of 9; sol. of 9 and enzyme; sol. of 9+ pamidronate; sol. of 9+ Zn²⁺; and sol. of 9+ Na₃VO₄ ([pamidronate]=[Zn²⁺]=[Na₃VO₄]=33 mM); row 2) pamidronate; row 3) Zn²⁺; and row 4) Na₃VO₄. (Left to right, Conc.=33; 3.3; 0.33; 0.033; 0.0033 mM).

FIG. 8. (8A) The solution of the hydrogelator at low concentration. (8B) Formation of hydrogels after adding surface-modified magnetic nanoparticles, abbreviated as “NP”. (8C) After applying magnetic field, represented as “H”, to the hydrogel for 1 hour. (8D) After applying magnetic field, H, to the hydrogel for 4 hours. (8E) After applying magnetic field, H, to the hydrogel for 10 hours.

FIG. 9. Chemical structures of the naphthalene containing dipeptide derivatives as the biocompatible hydrogelators.

FIG. 10. Frequency dependence of the storage moduli (G′: filled symbols) and the loss moduli (G″: open symbols) of hydrogels at the strain of 0.15% with concentrations at 0.5% of different hydrogels: ▪, 13; ●, 12; ▴, 11; and ▾, 14.

FIG. 11. TEM images of hydrogels formed by compound 11 (11A), compound 12 (11B), compound 13 (11C), and compound 14 (11D) with the concentration at 0.5 wt %.

FIG. 12. Chemical structures of the pentapeptide derivatives 15, 16, 17, 18, 19, and 20.

FIG. 13. Chemical structures of the β-aminoacid derivatives 21 and 22.

FIG. 14. Optical images of the hydrogels of 21 (14A) and 22 (14B).

FIG. 15. Gelation properties of the pentapeptides 15 (SEQ. ID No. 1), 16 (SEQ. ID No. 2), 17 (SEQ. ID No. 3), 18 (SEQ. ID No. 4), 19 (SEQ. ID No. 5), and 20 (SEQ. ID No. 6).

### DETAILED DESCRIPTION OF THE INVENTION

The present invention pertains to the design and application of a new type of supramolecular hydrogel having a three-dimensional, self-assembling, elastic, network structure comprising non-polymeric, functional molecules and a liquid medium, whereby said functional molecules are noncovalently crosslinked. The functional molecules (or entities) may be, for instance, anti-inflammatory molecules, antibiotics, metal chelators, anticancer agents, small peptides, surface-modified nanoparticles, or a combination thereof.

The antibiotics may be, for instance, vancomycin, penicillin, amoxicillin, cephalosporin, oxacillin, nafcillin, clindamycin, erythromycin, ciprofloxacin, rifampin, amphotericin, and/or sulfamethoxaole. The metal chelators may be chelating agents for radioactive isotopes, such as uranium chelating agents, cesium chelating agents, iodine chelating agents, stronium chelating agents, and/or americium chelating agents.

In an embodiment, said liquid medium is retained within the interstitial spaces of said structure. The liquid medium includes, but is not limited to, water, physiological saline, or other liquid medium. Examples of suitable liquid mediums have been identified so as to facilitate subsequent uses of the hydrogel.

The design of the supramolecular hydrogel includes: 1) modifying functional molecules to convert them into hydrogelators while enhancing or maintaining their therapeutic properties and 2) triggering the hydrogelation process, thereby resulting in the creation of a supramolecular hydrogel via formation of non-covalent crosslinks by the functional molecules.

The modification of step 1) includes attaching or removing one or more groups in the functional molecule. In step 2), the hydrogelation process may be triggered by physical, chemical, or enzymatic processes.

The present invention further provides a supramolecular hydrogel made by the above method.

Applications of the present invention include use of the supramolecular hydrogel, for instance, as a biomaterial for wound healing, tissue engineering, drug delivery, cell culture, and drug/inhibitor screening.

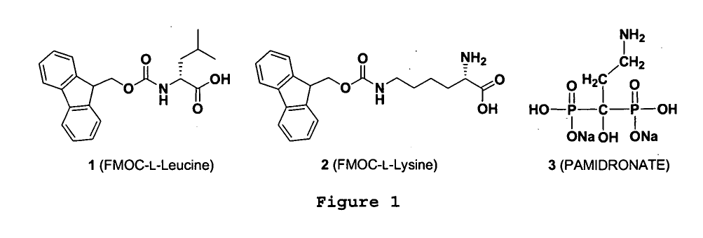

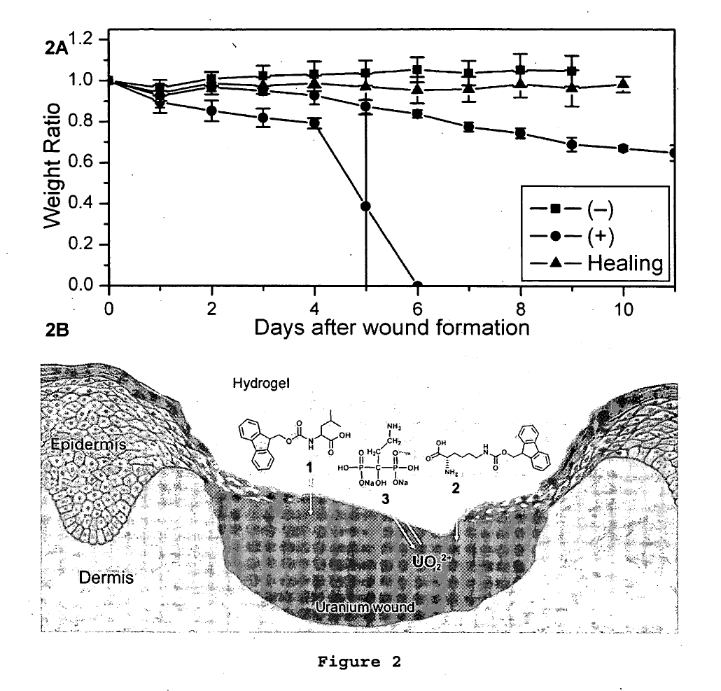

For example, in one embodiment, a multifunctional supramolecular hydrogel was designed so as to employ three small molecules 1, 2, and 3 (as shown in FIG. 1) as its structural components; two amino acid derivatives that can reduce inflammation; and a bisphosphonate that coordinates with UO₂²⁺ and lowers the toxicity of UO₂²⁺. These molecules self-assemble into networks of nanofibers as the matrices of the hydrogel. To demonstrate the in vivo activity of the supramolecular hydrogel, the hydrogel was administered topically on wound sites on the skin of mice that had been contaminated with uranyl nitrate. After being treated with the hydrogel, the mice recovered to normal, while the control group of mice (whose wounds were contaminated and untreated) weighed 35% less or expired (as shown in FIG. 2A). Notably, the results indicate that these small molecules maintained their therapeutic properties even when they served as the structural components of the supramolecular hydrogels, thus proving that supramolecular hydrogels can serve as a new type of biomaterial for a broad range of applications.

This invention provides a method of treating wounds, comprising the step of administering the above-discussed hydrogel to the external or internal wound of a patient in need thereof. In this instance, a particular medium adapted for treating wounds may be used.

Numerous of hydrogels based on polymeric hydrogelators have been developed in the art, with such hydrogels usually being mixed with therapeutic molecules so as to serve as drug delivery devices. However, several drawbacks are inherently associated with polymeric hydrogels: 1) polymeric hydrogelators, themselves, are normally passive (i.e., the polymers do not have therapeutic properties); 2) polymers have to be biodegradable; 3) the mixture of therapeutic agents and polymers is not entirely homogeneous (i.e., unwanted phase separation may occur), which may cause uncontrolled release of the drug molecules; and 4) the amount of therapeutic agents may be limited due to the use of polymers.

On the other hand, the present invention, directly uses the therapeutic or functional (non-polymeric) molecules as the hydrogelators, the desired properties of the hydrogels can be easily tailored. The term “non-polymeric” means that the molecules do not have covalently-linked repeating units. However, this invention does not exclude the use of polymers in combination of non-polymer.

Accordingly, for example, biodegradability and biocompatibility can be built into the molecules; drug molecules can be made to distribute more homogenously in the hydrogels; and large amount of drug molecules can be incorporated into the hydrogels. The hydrogel of the present invention can also form composites with magnetic nanoparticles. Such composites exhibit magnetoresponses, which may allow for controlled drug release via a magnetic field or a magnetic actuator.

The present invention also provides a enzyme inhibitor resulting from the screening method, which is not previously drawn.

The present invention additionally provides a method of culturing cells, comprising utilizing the previously discussed hydrogel as the three-dimensional matrix for cell growth.

In addition to the examples briefly mentioned above, below are additional examples, described in detail, pertaining to the present invention. The examples described herein are merely illustrative and are not intended to limit the present invention to such. One of ordinary skill in the art will be able to appreciate the full scope of the present invention and its equivalents in light of the teachings herein.

### EXAMPLE 1

### Wound Healing

To illustrate the biological activity of the supramolecular hydrogel of the present invention, the hydrogel, comprising the functional molecules shown in FIG. 1, was used to treat a uranium wound, which was created by scratching the skin on the back of mice and externally administering uranyl nitrate to the wound. The hydrogel was then topically administered to the wounds of the negative control group 20 minutes afterwards but not for the positive control group. The results of the experiment are shown in FIG. 2A. The mice in all groups exhibited initial weight loss the next day due to the effects of the wound. The negative control group recovered quickly from the wound after experiencing slight initial weight-loss and returned to normal growth on day 2. In contrast, the positive control group showed continuous weight-loss until expiration in about five days or 35% weight-loss over the next ten days. Thus, when the hydrogel was administered topically to the uranyl nitrate wounds of the mice in the negative control group, the mice experienced little weight loss and a nice recovery, with none of the toxic effects of the uranyl nitrate being observed in the mice's daily behavior.

FIG. 2B depicts the plausible delivery process of the functional molecules shown in FIG. 1. Both 1 and 2 migrate into the wound to reduce the inflammation by blocking the recruitment of neutropils into the inflamed site, and 3 decreases the toxicity of UO₂²⁺ by chelating with UO₂²⁺. In addition, since the hydrogel is able to “uptake” UO₂²⁺ from a uranyl nitrate solution, the hydrogel absorbs some of the UO₂²⁺ from the wound site and, thus, further reduces the damage caused by UO₂²⁺.

Although the effectiveness against a wound caused by other radioactive elements remains to be tested, the present hydrogel can be used advantageously in the confinement of radioactive uranium compared to liquid-based treatments since the hydrogel absorbs UO₂²⁺ well and has little fluidity. Thus, the hydrogel of the present invention is useful as an emergency treatment for uranium wounds. Accordingly, the above example demonstrates that other combinations of hydrogelators, selected from a pool of pharmaceutical molecules, may be used to create other useful biomaterials.

### EXAMPLE 2

### Noncovalent Crosslinking Supramolecular Hydrogels

Although in-situ polymerization allows enhanced stability of small-molecular gels, such a covalent cross-linking approach usually requires additional chemical synthesis, which alters the properties of the hydrogelators, and may result in the loss of biocompatibility and biodegradability. Accordingly, the use of molecular recognition (noncovalent crosslinking) to enhance the elasticity of the small-molecular hydrogels is preferred. For instance, the addition of a ligand into the mechanically-weak hydrogels of a derivative of the receptor leads to up to a million-fold increase in the storage modulus of the hydrogel. The term “noncovalent crosslinking” means that the crosslinking is realized by hydrogen bonding, hydrophobic forces, or ionic forces.

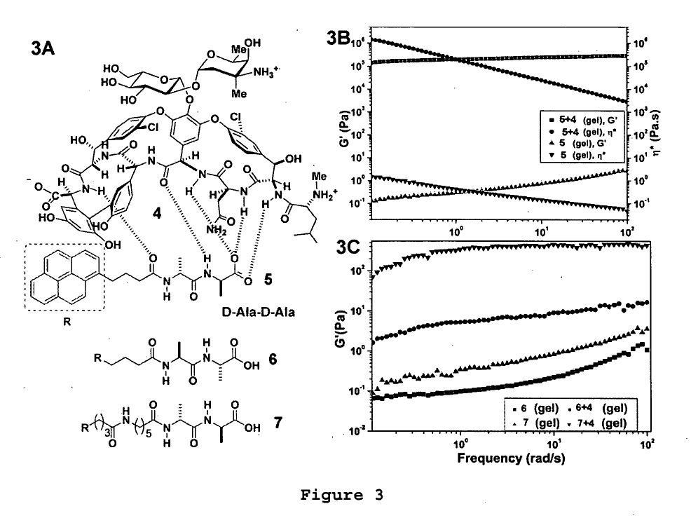

In one embodiment, vancomycin (Van) was selected as the ligand 4 and a D-Ala-D-Ala derivative was selected as the receptor 5 because of the well-established molecular recognition (FIG. 3A) between 4 and 5 in aqueous solution. Compound 5 gels water at the minimum gelation concentration of ˜30 mM and pH=9.5. In contrast, the mixture of 4 and 5 (mole ratio=1:1) forms a hydrogel at the minimum gel concentration of 5 mM and pH=9.5. Dynamic oscillatory measurements were used to evaluate the viscoelastic behavior of these two hydrogels at the same concentration (30 mM). To ensure that the hydrogels are reversible upon applying a shear force, all the frequency sweep measurements followed the determination of the linear viscoelastic regime by a strain sweep. As shown in the linear viscoelastic frequency sweep response of the hydrogels (FIG. 3B), the storage modulus (G′) of the hydrogel of 5 is 0.12 Pa at 0.1 rad/s. The frequency dependence versus complex viscosity (η*∝(frequency)ⁿ⁻¹, n=0.47±0.006) and a nonlinear frequency response started at 100 rad/s indicate that 5 can form only a liquid-like hydrogel. At the concentration of 30 mM, G′ of the hydrogel of 5+4 is 1.6×10⁵ Pa at 0.1 rad/s, and its frequency dependence versus complex viscosity (η*∝(frequency)ⁿ⁻¹, n=0.15±0.006) indicates the solid-like and highly elastic features of the hydrogel. Increasing the molar ratio of 4 (compared to 5) from zero to one increases G′ of the hydrogel of 5+4, following a power law (G′∝[4]ⁿ, n=5.93±0.31), suggesting that 4 acts as a crosslinker.

### EXAMPLE 3

### Antibiotic Supramolecular Hydrogels

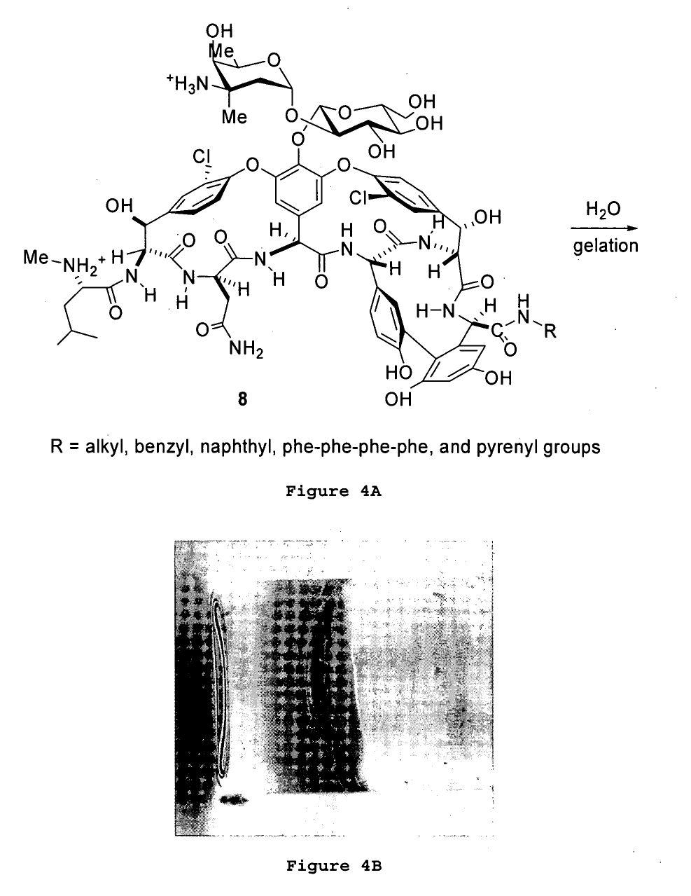

FIG. 4A shows the chemical structure of 8 (when R=pyrenyl), and FIG. 4B shows the picture of the hydrogel formed by adding 6.5 mg of 8 into 1.8 ml of water, corresponding to ˜0.36 wt % (2.2 mM) of the gelator and ˜23000 of water molecules/gelator molecule. 8 was unexpectedly potent (0.125 to 2 μg/ml, being 8 to 11 fold dilutions lower than the corresponding vancomycin) against VRE (2 vanA-positive Enterococcus faecalis, 4 vanA-positive E. faecium, 4 vanB-positive E. faecium). The strong tendency to self-assemble and the unexpected potency of 8 also lead us to speculate that 8 might aggregate into supramolecular structures at the cell surface when its local concentration is high

### EXAMPLE 4

### Enzymatic Formation of the Supramolecular Hydrogels

Recently, Messersmith, et al.² reported using an enzyme to crosslink polymers to induce hydrogelation, and Mooney, et al.³ demonstrated using cells as the crosslinkers for polymers to promote gelation. Both methods are believed to be advantageous in the biomedical application of hydrogels. Similar methodologies, however, have yet to be explored with hydrogels formed by small molecules. The term “small molecules” means molecules without covalently linked repeating units and includes small peptides (e.g., derivatives of single amino acids, dipeptides, tripeptides, β-aminoacids, and pentapetides, whereby the molecular weight of said derivatives are less than 3.0 KD). As used in the present disclosure, “small molecules” may be used interchangeably with “non-polymeric” molecules.

In the present invention, an enzymatic reaction was used to convert an ionic group on a derivative of an amino acid into a neutral group, which creates a small molecular hydrogelator and leads to the formation of a supramolecular hydrogel. This gelation process utilizes an alkaline phosphatase, one of the components of kinase/phosphatase switches that regulate protein activity, to dephosphorylate the PO₄³⁻ of N-(fluorenyl-methoxycarbonyl)tyrosine phosphate (9) under basic conditions. Unlike previously reported enzymatic gelation processes, this process, which involves bond breaking rather than bond formation, adjusts the balance of the hydrophobicity and hydrophilicity of the precursor, a simple amphiphilic derivative of amino acids, to yield a hydrogelator. Since dephosphorylation is a common, yet important, biological reaction existing in many organisms, its coupling with hydrogelation provides an advantageous way of generating and utilizing biomaterials based on supramolecular hydrogels.

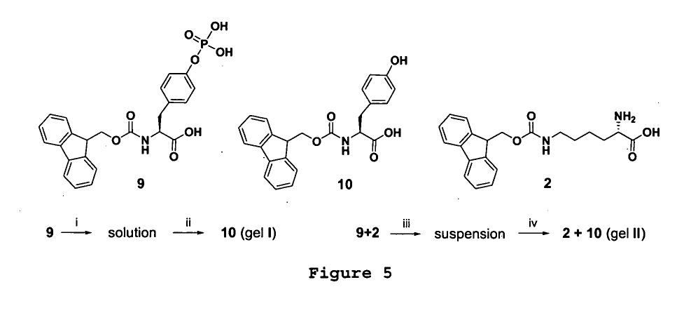

FIG. 5 illustrates two typical procedures for inducing gelation by dephosphorylation of 9. In the first case, 9 and one equivalent Na₂CO₃ is dissolved in a phosphate buffer (pH=9.6) to form a clear solution. The addition of alkaline phosphatase converts the solution of 9 into an opaque hydrogel of 10 with pH of 9.6 at 37° C. in 30 min.

In the second case, equal moles of 9 and 2 and two equivalents of Na₂CO₃ are mixed in the phosphate buffer (pH=9.6) to form a suspension upon gentle heating. The suspension is then added to the alkaline phosphatase and kept at ˜60° C. for three minutes. The suspension turns into a clear solution, which forms a clear hydrogel upon cooling to room temperature. When the same two procedures were repeated without the addition of the alkaline phosphatase, neither procedure led to the formation of hydrogels.

### EXAMPLE 5

### Using Supramolecular Hydrogels to Screen the Inhibitor of Enzymes

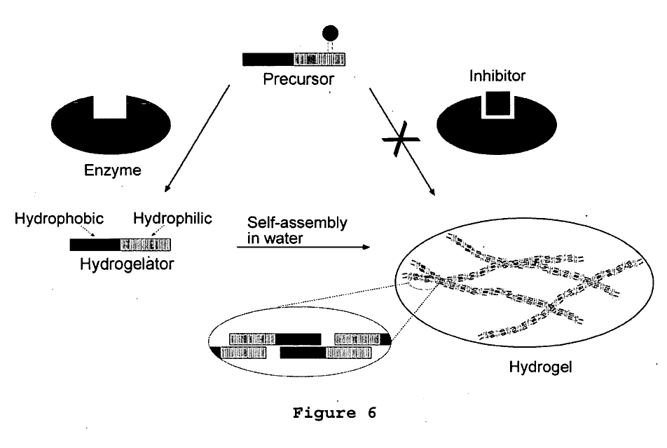

FIG. 6 illustrates the design of the visual assay. The precursor, which acts as the substrate of an enzyme, transforms into a hydrogelator when the enzyme catalyzes its conversion. Then, the self-assembly of the hydrogelators in water induces the formation of hydrogel. When inhibitors competitively bind with the active site of the enzyme and block the conversion of the precursor catalyzed by the enzyme, no hydrogel forms. Therefore, the macroscopic solution-to-gel transition (which can be observed visually) of the solution of the precursor reports the inactivation of the enzyme by the inhibitors.

This approach has a unique feature—it enlists water molecules as part of the reporting system. In addition, no spectrometer is required for observing the solution-to-gel phase transition. This simple and inexpensive method may be useful, not only for screening the inhibitors but also, for detecting the presence of enzymes when appropriate precursors are used. To verify the feasibility of the design shown in FIG. 6, a simple amino acid derivative (9), which can be converted into a hydrogelator (10) by dephosphorylation, was used to screen the inhibitors for an acid phosphatase.

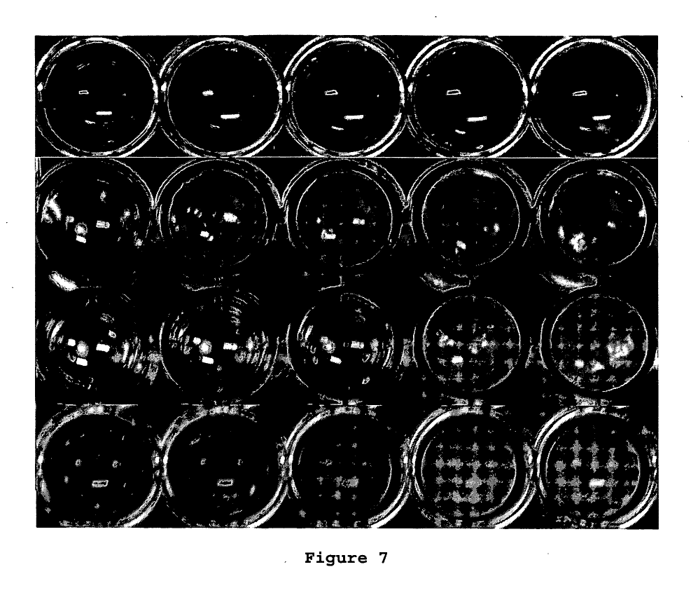

Since the acid phosphatase catalyzes the conversion of 9 to 10 and leads to hydrogelation at a pH=6.0 and 37° C., the event of hydrogelation can indicate the activity of inhibitors for the acid phosphatase itself. Pamidronate disodium, Zn²⁺, and sodium orthovanadate (Na₃VO₄) were chosen to estimate their minimum inhibition concentrations for the acid phosphatase. The three compounds were first mixed with the enzyme at a series of concentrations, respectively, followed by the addition of 9 to the solutions 10 minutes after mixing. After an additional 30 minutes of incubation, the solution-to-gel phase transition indicates the minimum inhibition concentration of the compounds. From the changes of rows 2, 3, and 4 in FIG. 7, the minimum inhibition concentrations of Pamidronate disodium, Zn²⁺, and sodium orthovanadate (Na₃VO₄) for the acid phosphatase were determined to be 33 mM, 0.33 mM, and 3.3 mM, respectively. This result corresponds closely to the literature values for this enzyme, thus validating our design.

### EXAMPLE 6

### Magnetoresponse of the Supramolecular Hydrogels

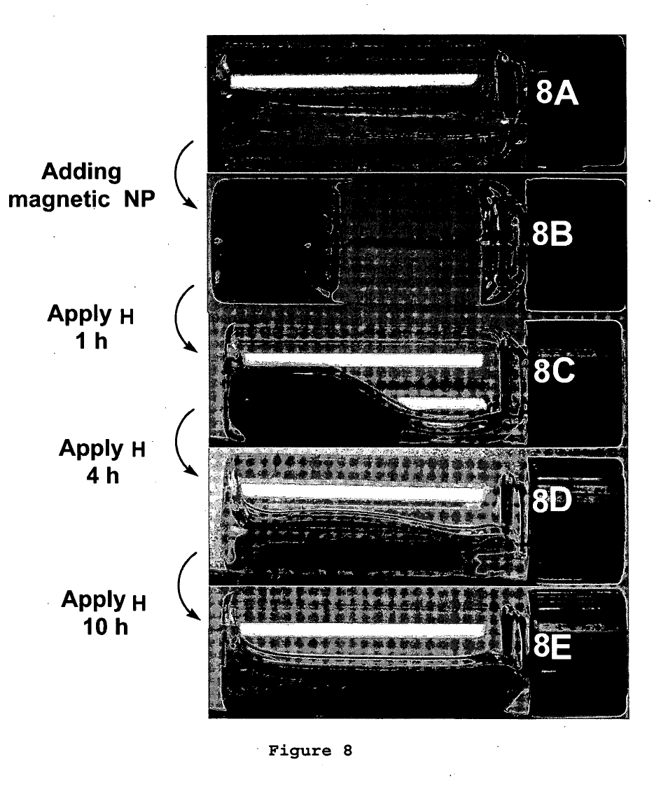

FIG. 8 shows the formation of the magnetic responsive hydrogel (FIG. 8B) after adding surface-modified magnetic nanoparticles into the solution of the diluted hydrogelator (FIG. 8A). After applying a small magnetic field to the hydrogel constantly for 10 hours (FIG. 8E), the hydrogel transforms into a solution and the aggregate of magnetic nanoparticles (for example, iron oxide). This process can be used to trigger the release of a drug from the hydrogel by a magnetic force.

### EXAMPLE 7

### Hydrogelators of Naphthalene-containing Dipeptides

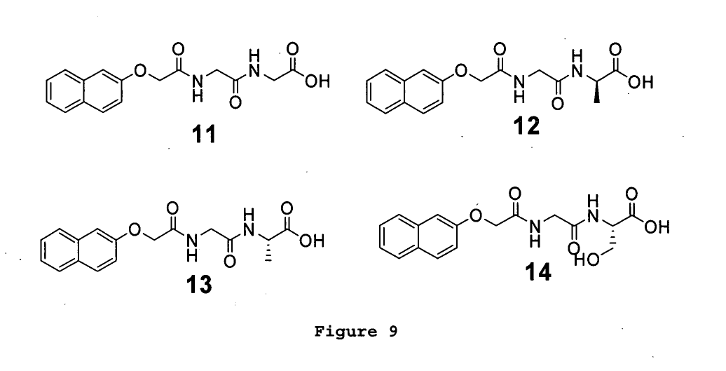

Hydrogelators can be made more biocompatible by containing a naphthalene group, a common fragment in drug molecules. FIG. 9 shows the chemical structures of the naphthalene-containing dipeptides that are hydrogelators. The syntheses of compounds 11, 12, 13, and 14 were based on 2-(naphthalen-2-yloxy)acetic acid. The syntheses of 11-14 were quite simple, just requiring the use of an active ester of N-hydroxy succinimine to react with different amino acids, and the overall yields were relatively high (60-80%).

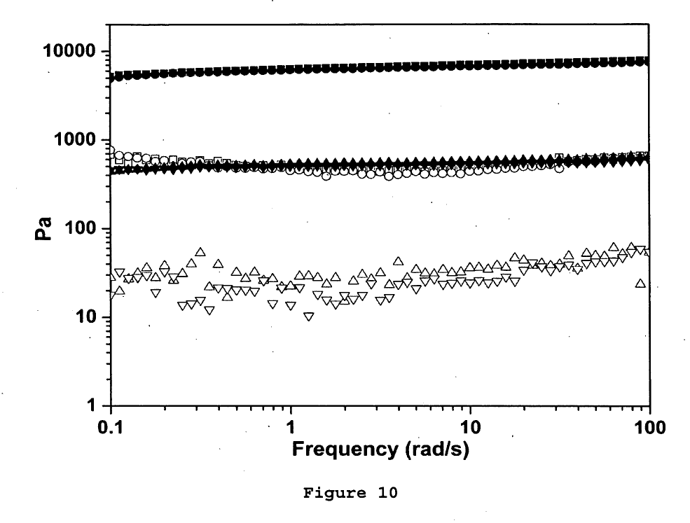

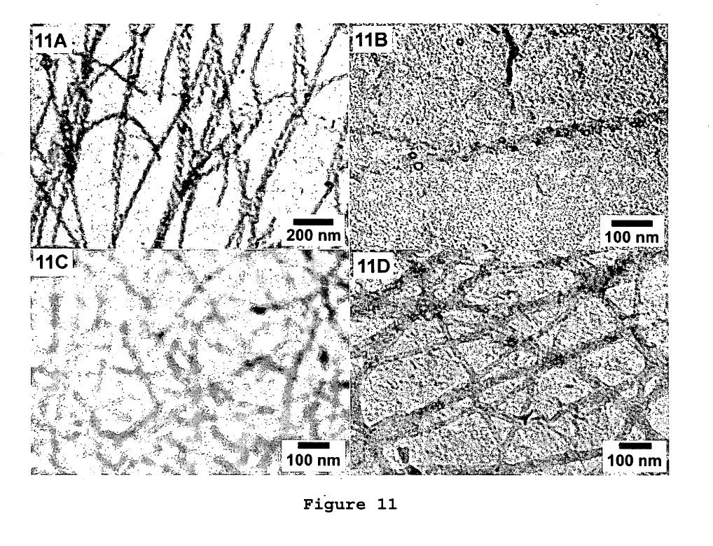

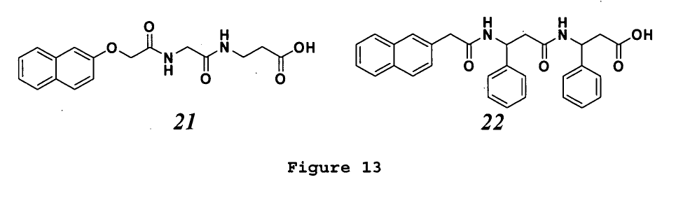

Compound 11-14 showed excellent abilities to gel water at pH 2 and could form gels with concentrations of <0.10 wt %. Compounds 12 and 13 were the best gelators and could gel water at a concentration of 0.07 wt %. Compounds 11 and 14 exhibited similar behaviors of gelation to 2 and 3, except at higher concentrations ([11]=0.10 wt % and [14]=0.08 wt %). FIG. 10 shows the linear viscoelastic frequency sweep response of the four as-prepared hydrogels. All of them exhibited very weak frequency dependence from 0.1 to 100 rad/s, with G′ dominating G″, which means that they are effectively hydrogels. FIG. 11 displays the transmission electron micrographs (TEM) of the hydrogels, which reveals that the hydrogels made from 12 (FIG. 11B) or 13 (FIG. 11C) containing helical structures with very uniform size of about 30 nm and pitchs of about 60 nm. These results demonstrated that naphthalene moiety is an effective hydrogelation promoter.

### EXAMPLE 8

### Hydrogelators of Pentapeptide Derivatives

In order to explore pentapeptide-based hydrogels as potential biomaterials, three aromatic moieties (pyrene (P), fluorene (F), and naphthalene (N)) were covalently linked to a series of pentapeptides: GAGAS, SEQ ID No. 1, (15), GVPVP, SEQ ID No. 2, (16), VPGVG, SEQ ID No. 3, (17), VTEEI, SEQ ID No. 4 (18), VYGGG, SEQ ID No. 5, (19), and YGFGG, SEQ ID No. 5 (20). The balance of intermolecular aromatic-aromatic interactions and hydrogen bonds of these molecules can lead to their self-assemblies in water, which provide matrices of nanofibers for hydrogelation.

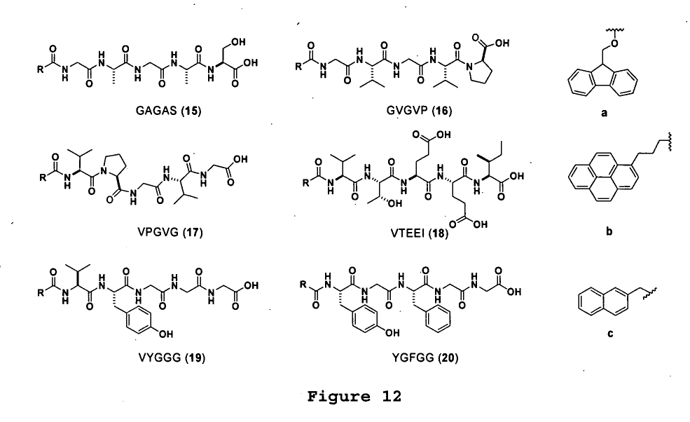

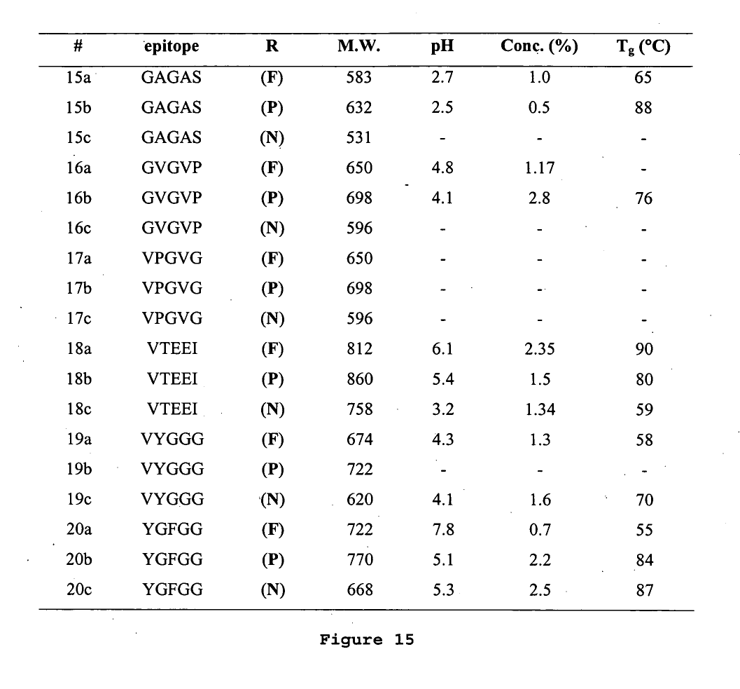

All the pentapeptides (structures shown in FIG. 12) were prepared by solid-phase synthesis using 2-chlorotrityl resin and the corresponding N^{α}-Fmoc protected amino acids with side chains properly protected by a t-butyl group. The first amino acid at C-terminal was loaded on the resin, followed by removal of the Fmoc group. Then the next Fmoc-protected amino acid was coupled with the free amino group using TBTU/HOBt as the coupling reagent. Finally, the N-terminus of the pentapeptides were either protected by Fmoc or coupled with 1-pyrenebutyric acid or 1-naphthalen acetic acid to afford the hydrophobic group. Upon completion of all the coupling, the pentapeptides were cleaved from the resin by trifluoroacetic acid (TFA) with 2.5% triisopropylsilane and 2.5% water as scavenger and purified by reverse phase HPLC. Gelation properties of the pentapeptides are shown in FIG. 15.

Most of the compounds can gel water under appropriate pH. When the pH becomes higher than the listed value, the gel tends to become a clear solution, while a lower pH always leads to precipitation rather than homogeneous gel formation. GAGAS, the epitope with the least bulk side chains, appears to be quite hydrophilic in water. Naphthalene seems not to be hydrophobic enough to keep the hydrophobic/hydrophilic balance needed for Naph-GAGAS to gel water since Naph-GAGAS is soluble in water even under low pH and high concentration. With a more hydrophobic group, Fmoc-GAGAS and Pyrene-GAGAS become hydrogelators which can gel water under quite acidic conditions. GVGVP, with larger side-chains in valine and a proline at the end of the peptide chain, shows poor solubility in water. However, it is still not a good candidate as a hydrophilic tail in a hydrogelator. Only Pyrene-GVGVP can form gel easily.

Hydrogel by Fmoc-GVGVP can be obtained by carefully adjusting the pH to 4.8, with the hydrogel not being thermal-reversible. Naph-GVGVP either dissolves in water at a pH higher than 4 or becomes a suspension at a lower. pH. Upon heating, it also melts. All three compounds, with VPGVG as the hydrophilic part, fail to gel water at the tested condition. They all show sharp solubility changes with pH and low melting points. VTEEI, in which all the five amino acids have large side chains, shows a satisfactory ability to gel water when attached to Fmoc, pyrene or naphthalene. Notably, epitope VYGGG, Fmoc, and naphthalene are appropriate hydrophobic groups for forming hydrogels while pyrene appears to be so hydrophobic that Pyrene-VYGGG is insoluble in water even under basic conditions. These examples demonstrate that pentapetides can be converted into excellent hydrogelators for generating supramolecular hydrogels as potential biomaterials.

### EXAMPLE 9

### Hydrogelators of β-amino Acid Derivatives

Being used in vivo, oligopeptide-based scaffolds are biodegradable because proteolytic enzymes in biological systems will catalyze their hydrolysis.⁴ Such a inherent suseptibilty towards enzymes shortens the in vivo lifetime of these peptide-based hydrogels, reduces their efficacy, and limits their scope of applications when long-term bioavailability is required. On the other hand, the disadvantage of proteolysis is a common feature for peptide-based therapeutic agents. Therefore, many efforts have focused on designing and synthesizing non-peptide molecules that mimic the functions of peptides or proteins to achieve prolonged or controlled stability and bioavailability of those molecules.⁴

Among the peptidomimics,⁵ β-peptides, which contain β-amino acids, have received intensive attention due to their improved biostability.^{4, 6-10} Despite the rapid progress in the designing and synthesis of β-peptides, the application of β-amino acids for controlling the bioavailability of supermolecular hydrogels remains unexplored since it is unknown if a β-amino acid derivative will act as a hydrogelator. FIG. 13 illustrates the chemical structures of the two hydrogelators 21 and 22, which are dipeptidic mimics linked with naphthalene groups via amide bonds. The synthesis of both compounds is simple and straighforward: the N-hydroxy succinimide (NHS) activated ester of 2-(naphthalen-2-yloxy)acetic acid or 2-(naphthalen-2-yl)acetic acid react with glycine or β³-phenylalanine to afford 2-(2-(naphthalen-2-yloxy)acetamido)acetic acid or 3-(2-(naphthalen-2-yl)acetamido)-3-phenylpropanoic acid, respectively. The subsequent NHS assisted coupling gives 21 in 67% yield, and 22 in 72% yield.

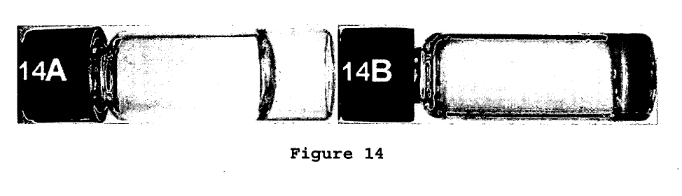

After 5 mg of 1 is suspended in 1.0 mL of water, the adjustment of the pH value of the suspension to 4.8 results in a clear solution, which provides a transparent hydrogel (FIG. 14A). Similarly, 5 mg of 2 in 1.0 mL of water also can form an slightly opaque hydrogel (FIG. 14B) by adjusting the pH or temperature. The confirmation of β-amino acids-based hydrogelators should provide a new way to tailor the stability of hydrogels in a biological enviroment and ultimately expand the ranges of applications of the hydrogels as biomaterials.

### REFERENCES

1. Burch, R. M.; Weitzberg, M.; Blok, N.; Muhlhauser, R.; Martin, D.; Farmer, S. G.; Bator, J. M.; Connor, J. R.; Ko, C.; Kuhn, W.; McMillan, B. A.; Raynor, M.; Shearer, B. G.; Tiffany, C.; Wilkins, D. E., N-(Fluorenyl-9-Methoxycarbonyl) Amino-Acids, a Class of Antiinflammatory Agents with a Different Mechanism of Action. Proceedings of the National Academy of Sciences of the United States of America 1991, 88, (2), 355-359.

2. Hu, B.-H.; Messersmith, P. B., Rational Design of Transglutaminase Substrate Peptides for Rapid Enzymatic Formation of Hydrogels. J. Am. Chem. Soc. 2003, 125, (47), 14298-14299.

3. Lee, H. Y.; Kong, H. J.; Larson, R. G.; Mooney, D. J., Adv. Mater. 2003, 15, 1828-1832.

4. Seebach, D.; Matthews, J. L., beta-peptides: a surprise at every turn. Chemical Communications 1997, (21), 2015-2022.

5. Giannis, A., PEPTIDOMIMETICS FOR RECEPTOR LIGANDS DISCOVERY, DEVELOPMENT, AND MEDICAL PERSPECTIVES. Angew. Chem. Intl. Ed. 1993, 32, (9), 1244-1267.

6. Appella, D. H.; Christianson, L. A.; Karle, I. L.; Powell, D. R.; Gellman, S. H., beta-peptide foldamers: Robust Helix formation in a new family of beta-amino acid oligomers. Journal of the American Chemical Society 1996, 118, (51), 13071-13072.

7. Seebach, D.; Abele, S.; Gademann, K.; Guichard, G.; Hintermann, T.; Jaun, B.; Matthews, J. L.; Schreiber, J. V., beta(2)- and beta(3)-peptides with proteinaceous side chains: Synthesis and solution structures of constitutional isomers, a novel helical secondary structure and the influence of salvation and hydrophobic interactions on folding. Helvetica Chimica Acta 1998, 81, (5), 932-982.

8. Hook, D. F.; Bindschadler, P.; Mahajan, Y. R.; Sebesta, R.; Kast, P.; Seebach, D., The proteolytic stability of ‘designed’ beta-peptides containing alpha-peptide-bond mimics and of mixed alpha,beta-peptides: Application to the construction of MHC-binding peptides. Chemistry & Biodiversity 2005, 2, (5), 591-632.

9. Martinek, T. A.; Fulop, F., Side-chain control of beta-peptide secondary structures—Design principles. European Journal of Biochemistry 2003, 270, (18), 3657-3666.

10. Porter, E. A.; Wang, X. F.; Lee, H. S.; Weisblum, B.; Gellman, S. H., Antibiotics—Non-haemolytic beta-amino-acid oligomers. Nature 2000, 404, (6778), 565-565.

1. A supramolecular hydrogel having a three-dimensional, self-assembling, elastic, network structure comprising non-polymeric, functional molecules and a liquid medium, whereby said functional molecules are noncovalently crosslinked.

2. The hydrogel of claim 1, wherein the functional molecules are selected from the group consisting of anti-inflammatory molecules, antibiotics, metal chelators, anticancer agents, small peptides, surface-modified magnetic nanoparticles, and a combination thereof.

3. The hydrogel of claim 2, wherein the small peptides are selected from the group consisting of the derivatives of single amino acids, dipeptides, tripeptides, β-amino acids, and pentapetides, whereby the molecular weight of said derivatives are less than 3.0 KD.

4. The hydrogel of claim 2, wherein the anti-inflammatory molecules are selected from the group consisting of N-(Fluorenyl-9-methoxycarbonyl)-L-Leucine and N-(Fluorenyl-9-methoxycarbonyl)-L-Lysine.

5. The hydrogel of claim 2, wherein the antibiotics are selected from the group consisting of vancomycin, penicillin, amoxicillin, cephalosporin, oxacillin, nafcillin, clindamycin, erythromycin, ciprofloxacin, rifampin, amphotericin, and sulfamethoxaole.

6. (canceled)

7. The hydrogel of claim 2, wherein the metal chelators are selected from the group consisting of uranium chelating agents, cesium chelating agents, iodine chelating agents, stronium chelating agents, and americium chelating agents.

8. The hydrogel of claim 7, wherein the uranium chelating agent is a bisphosphonate.

9. The hydrogel of claim 8, wherein the bisphosphonate is pamidronate.

10. The hydrogel of claim 1, wherein the noncovalent crosslinking is effectuated by ligand-receptor interactions.

11. (canceled)

12. The hydrogel of claim 10, wherein the ligand is vancomycin and the receptor is a D-Ala-D-Ala derivative.

13. (canceled)

14. A method of treating wounds, comprising the step of administering the hydrogel of claim 1 to the external or internal wound of a patient in need thereof.

15. The method of claim 14, wherein the wound is contaminated with radioactive isotopes.

16. The method of claim 15, wherein the radioactive isotopes are selected from the group consisting of uranyl nitrate, uranium oxide, and uranium.

17. A method of making a supramolecular hydrogel, comprising the use of a precursor of hydrogelator that is subsequently hydrolyzed by a hydrolyase under proper conditions, thereby resulting in the formation of said hydrogel.

18. A method of claim 17, comprising dephosphorylation of N-(fluorenylmethoxycarbonyl) tyrosine phosphate with an alkaline phosphatase under basic conditions, thereby resulting in the formation of said hydrogel.

19. The method of claim 18, wherein dephosphorylation comprises the steps of:
a. Dissolving N-(fluorenylmethoxycarbonyl) tyrosine phosphate and one equivalent of Na₂CO₃ in a phosphate buffer to form a solution;
b. Adding alkaline phosphatase to the solution; and
c. Maintaining the solution at a temperature of about 37° C.

20. The method of claim 18, wherein dephosphorylation comprises the steps of:
a. Mixing equal moles of N-(fluorenylmethoxycarbonyl)tyrosine phosphate and N-(Fluorenyl-9-methoxycarbonyl)-L-Lysine and two equivalents of Na₂CO₃ in a phosphate buffer to form a suspension upon heating;
b. Adding alkaline phosphatase to the suspension; and
c. Maintaining the suspension at a temperature of about 60° C.

21. (canceled)

22. A method of making a supramolecular hydrogel, comprising the steps of:
a. Modifying functional molecules to convert them into hydrogelators while enhancing or maintaining their therapeutic properties and
b. Triggering the hydrogelation process by enzymatic processes, thereby resulting in the creation of a supramolecular hydrogel via formation of noncovalent crosslinks by said functional molecules.

23. The supramolecular hydrogel made by the method of claim 17.

24. (canceled)

25. (canceled)

26. A method of culturing cells, comprising the use of the hydrogel of claim 1 as the three-dimensional matrix for cell growth.


SEQUENCE LISTING (6 sequences)

SEQ ID NO: 1 - protein, 5 aa, Artificial
  Synthetic
GAGAS

SEQ ID NO: 2 - protein, 5 aa, Artificial
  Synthetic
GVPVP

SEQ ID NO: 3 - protein, 5 aa, Artificial
  Synthetic
VPGVG

SEQ ID NO: 4 - protein, 5 aa, Artificial
  Synthetic
VTEEI

SEQ ID NO: 5 - protein, 5 aa, Artificial
  Synthetic
VYGGG

SEQ ID NO: 6 - protein, 5 aa, Artificial
  Synthetic
YGFGG